(19) United States
(12) Patent Application Publication
(10) Pub. No.: US 2011/0091400 A1
(43) Pub. Date: Apr. 21, 2011

(54) COSMETIC EMULSIFIER COMBINATION

(75) Inventors: Mirko Tesch, Hamburg (DE); Kerstin Skubsch, Prisdorf (DE); Alexandra Blohm, Hamburg (DE)

(73) Assignee: BEIERSDORF AG, Hamburg (DE)

(21) Appl. No.: 12/672,355

(22) PCT Filed: Aug. 1, 2008
(86) PCT No.: PCT/EP08/06367

§ 371 Date: May 5, 2010

(30) Foreign Application Priority Data
Aug. 9, 2007 (DE) 10 2007 038 413.2

(51) Int. Cl. A61K 8/92; A61Q 17/04

(52) U.S. Cl. 424/60; 514/547; 424/59

(57) ABSTRACT

The invention relates to a cosmetic composition comprising a) sodium stearyl glutamate, b) a crosslinked polymer of vinylpyrrolidone and acrylic acid.

The present invention relates to a cosmetic emulsion.

The desire to look beautiful and attractive is naturally rooted in people. Even if the ideal of beauty has undergone changes over the course of time, the desire for a flawless outward appearance has always been the aim of mankind. A significant part of a beautiful and attractive outward appearance is the condition and appearance of the skin.

So that the skin can completely fulfill its biological functions, it requires regular cleaning and care. Cleaning the skin serves here to remove dirt, sweat and remains of dead skin particles, which form an ideal breeding ground for pathogens and parasites of all types. Skincare products mostly serve to moisturize and regrease the skin. Active ingredients are often added to them which are intended to regenerate the skin and, for example, prevent and reduce its premature aging (e.g. the formation of wrinkles, lines).

Skin care products generally consist of emulsions. Emulsions are generally understood as meaning heterogeneous systems which consist of two liquids that are immiscible or only miscible with one another to a limited degree, these generally being referred to as phases, and in which one of the two liquids is dispersed in the form of very fine droplets in the other liquid. Viewed from the outside and with the naked eye, emulsions appear homogeneous.

In order to keep emulsions stable over an extended period and to prevent separation of the phases, so-called emulsifiers are added to the emulsions. The emulsifiers are generally molecules with a polar, hydrophilic structural element and a nonpolar, lipophilic structural element. At the end of the 1940s, a system was developed which was intended to facilitate the choice of emulsifiers. Each emulsifier is assigned a so-called HLB value (a dimensionless number between 0 and 20), which indicates whether a preferred water or oil solubility is present. Numbers below 9 characterize oil-soluble, hydrophobic emulsifiers, numbers above 11 water-soluble, hydrophilic emulsifiers. Emulsifiers with a particularly high HLB value (greater than 13) can also be used as washing-active surfactants.

Emulsions of the prior art have the disadvantage of being only electrolyte-stable to a limited degree. In the dissolved state, electrolytes alter the polarity of the two phases, in particular that of the aqueous phase, and therefore make an emulsion unstable (thermally unstable, storage-unstable, transport-unstable). The instability leads here to oil deposits and phase separations. Moreover, the use of electrolytes in emulsions causes their viscosity to change.

It was therefore the object of the present invention to develop an emulsifier system which is characterized by high tolerance to the electrolyte content and the type of electrolytes used. The emulsion to be developed should here be able to tolerate varying (including very large) amounts of electrolyte without becoming unstable.

Moreover, it was the object of the present invention to develop an emulsion whose viscosity can be adjusted without problems irrespective of the electrolyte content of the preparation over a wide viscosity range (from thin-liquid to cream-like viscous).

It was a further object of the present invention to develop a particularly stable and sensorily attractive emulsifier system.

Last but not least, it was the object of the present invention to develop a particularly simple emulsifier system which can be prepared in a cost-effective manner.

Surprisingly, the objects are achieved by a cosmetic preparation comprising

a) sodium stearyl glutamate,
b) a crosslinked polymer of vinylpyrrolidone and acrylic acid.

The preparations according to the invention are characterized by a particularly light skin feel that is not very greasy. Moreover, the preparations according to the invention are absorbed into the skin surprisingly rapidly.

Although the person skilled in the art is aware of EP 0 766 959, this specification was unable to point the way to the present invention.

In particular, it was surprising for the person skilled in the art that in the case of thin-liquid formulations according to the invention with a viscosity of e.g. 500 mPas, the preparations are, despite a high electrolyte content, stable at elevated temperature over a prolonged period and can be sprayed in a very fine form.

A further surprising and particularly advantageous property of thin-liquid preparations according to the invention is the sprayability “upside-down”, i.e. the consumer can easily apply the emulsion to the entire body from various positions. For this purpose, there are spray pumps e.g. from Seaquist which permit this type of application (PZ2 190DL USD1)

The following formulations do not fall within the scope of the present invention:

| | 1 | 2 | 3 | 4 |
|---|---|---|---|---|
| Glyceryl stearate | | | 2 | |
| Sodium stearoyl glutamate | 0.05 | 0.1 | 0.2 | 0.5 |
| Stearyl alcohol | | | 2 | |
| Copolymer of vinylpyrrolidone and acrylic | 2 | 1.6 | 0.5 | 1 |
| acid | | | | |
| Carbomer | | 0.2 | | |
| C₁₂₋₁₅ Alkyl benzoate | | 5 | 1 | 10 |
| Butylene glycol dicaprylate/dicaprate | 2 | 8 | | 10 |
| Phenethyl benzoate | | | 2 | 5 |
| Octyldodecanol | | 2 | 4 | |
| Myristyl myristate | | | | 1 |
| Caprylic/capric triglyceride | | | 2 | |
| Isodecyl neopentanoate | 3 | | | |
| Cyclomethicone | 2 | | | |
| Dimethicone | 2 | | | |
| Glycerol | 10 | 2 | 5 | 8 |
| Bis-ethylhexyloxyphenol methoxyphenyl | | 1 | | 2 |
| triazine | | | | |
| Butylmethoxydibenzoylmethane | | 4 | | 3 |
| Polysilicone-15 | | | 2 | |
| Ethylhexyl methoxycinnamate | | 3 | | |
| Octyl salicylate | | 3 | 2 | |
| Phenylbenzimidazole sulfonic acid | 1 | 0.5 | 1 | 2.5 |
| Phenylene-1,4-bis(2-benzimidazyl)-3,3′,5,5′- | 1 | | 3 | 0.5 |
| tetrasulfonic acid | | | | |
| Ethylhexyl triazine | 2 | | | 3 |
| Titanium dioxide | 2 | | | |
| Aluminum starch octenylsuccinate | 2 | | | 2 |
| Sodium starch octenylsuccinate | | | 3 | |
| Tapioca starch | 2 | | | |
| Alcohol | | | 4 | |
| Folic acid | | 0.03 | 0.02 | |
| Q 10 | 0.05 | | | |
| Na₂H₂EDTA | 0.2 | 0.2 | 0.2 | 0.2 |
| Perfume, preservative | q.s. | q.s. | q.s. | q.s. |
| Dyes, etc. | q.s. | q.s. | q.s. | q.s. |
| Sodium hydroxide | q.s. | q.s. | q.s. | q.s. |
| Water | ad 100.0 | ad 100.0 | ad 100.0 | ad 100.0 |

According to the invention, it is advantageous if the preparation according to the invention is present in the form of a O/W emulsion.

According to the invention, it is advantageous if the crosslinked polymer of vinylpyrrolidone and acrylic acid used is the compound with the CAS No: 527685-31-0.

According to the invention, crosslinked polymer of vinylpyrrolidone is used here advantageously in an amount of from 0.05 to 4% by weight and preferably in an amount of from 0.1 to 3% by weight, based on the total weight of the preparation.

According to the invention, the preparation according to the invention advantageously comprises sodium stearyl glutamate in a concentration of from 0.05 to 2% by weight and according to the invention preferably in a concentration of from 0.1 to 1% by weight, in each case based on the total weight of the preparation.

The sodium stearyl glutamate according to the invention can be obtained for example under the name Eumulgin SG from Cognis.

According to the invention, it is advantageous if the preparation according to the invention comprises sucrose polystearate.

According to the invention, sucrose polystearate is used here advantageously in an amount of from 0.1 to 6% by weight and preferably in an amount of from 0.2 to 4% by weight, based on the total weight of the preparation.

Embodiments of the present invention advantageous according to the invention are characterized in that the preparation comprises hydrogenated polyisobutene.

According to the invention, hydrogenated polyisobutene is used here advantageously in an amount of from 0.05 to 2% by weight and preferably in an amount of from 0.1 to 1% by weight, based on the total weight of the preparation.

According to the invention, it is advantageous if the preparation comprises electrolytes.

Within the context of the present invention, the electrolytes include organic and inorganic salts.

According to the invention, it is advantageous here if the salts contain sodium, potassium, triethanolammonium, ammonium, calcium, magnesium ions as cations.

As anions of the salts, it is possible to use, for example, chlorides, phosphates, carbonates, bicarbonates, sulfates, hydrogensulfates, citrates, lactates, EDTA, salicylates, ascorbates, acetates, benzoates and further carboxylic acid anions.

According to the invention, it is advantageous here if the electrolyte content is from 0.05 to 10% by weight and preferably from 0.1 to 5% by weight, based on the total weight of the preparation.

According to the invention, it is preferred if the preparation comprises one or more UV filters selected from the group phenylene-1,4-bis(2-benzimidazyl)-3,3′,5,5′-tetrasulfonic acid salts; 2-phenylbenzimidazole-5-sulfonic acid salts; 4-(2-oxo-3-bornylidenemethyl)benzene sulfonic acid salts; 2-methyl-5-(2-oxo-3-bornylidenemethyl)sulfonic acid salts; terephthalidene dicamphor sulfonic acid salts.

According to the invention, phenylene-1,4-bis(2-benzimidazyl)-3,3′5,5′-tetrasulfonic acid salts are advantageously used in an amount of from 0.1 to 10% by weight and preferably in an amount of from 0.5 to 5% by weight, based on the total weight of the preparation.

According to the invention, phenylbenzimidazole-5-sulfonic acid salts are used here advantageously in an amount of from 0.1 to 8% by weight and preferably in an amount of from 0.5 to 5% by weight, based on the total weight of the preparation.

According to the invention, terephthalidene dicamphor sulfonic acid salts are used here advantageously in an amount of from 0.1 to 10% by weight and preferably in an amount of from 0.5 to 5% by weight, based on the total weight of the preparation.

Embodiments of the present invention advantageous according to the invention are characterized in that the preparation comprises UV filters selected from the group of the compounds 2-(2H-benzotriazol-2-yl)-4-methyl-6-[2-methyl-3-[1,3,3,3-tetramethyl-1-(tri-methylsilyl)oxy]disiloxanyl)propyl]phenol; 3-(4-methylbenzylidene)camphor; 3-benzylidene-camphor; ethylhexyl salicylate; 2-ethylhexyl 4-(dimethylamino)benzoate; amyl 4-(dimethylamino)benzoate; di(2-ethylhexyl) 4-methoxybenzalmalonate; 2-ethylhexyl 4-methoxycinnamate; isoamyl 4-methoxycinnamate; 2-hydroxy-4-methoxybenzophenone, 2-hydroxy-4-methoxy-4′-methylbenzophenone; 2,2′-dihydroxy-4-methoxybenzophenone; hexyl 2-(4′-diethylamino-2′-hydroxybenzoyl)benzoate, 4-(tert-butyl)-4′-methoxydibenzoyl-methane; homomethyl salicylate; 2-ethylhexyl 2-hydroxybenzoate; 2-ethylhexyl 2-cyano-3,3-diphenylacrylate; dimethicodiethyl benzalmalonate; 2-ethylhexyl 2-cyano-3,3-diphenylacrylate; 3-(4-(2,2-bis-ethoxycarbonylvinyl)phenoxy)propenyl)methoxysiloxane/-dimethylsiloxane copolymer; dioctylbutylamidotriazone (INCI: Diethylhexyl Butamidotriazone); 2,4-bis[5-1 (dimethylpropyl)benzoxazol-2-yl(4-phenyl)imino]-6-(2-ethylhexyl)imino-1,3,5-triazine with the (CAS No. 288254-16-0); tris(2-ethylhexyl) 4,4′,4″-(1,3,5-triazine-2,4,6-triyltriimino)trisbenzoate (also: 2,4,6-tris[anilino(p-carbo-2′-ethyl-1′-hexyloxy)]-1,3,5-triazine (INCI: Ethylhexyl Triazone); 2,4-bis{[4-(2-ethylhexyloxy)-2-hydroxy]phenyl}-6-(4-methoxyphenyl)-1,3,5-triazine; 4-(tert-butyl)-4′-methoxydibenzoyl-methane; 4-dicyanomethylene-2,6-dimethyl-1,4-dihydropyridine N-(ethyloxysulfate ester salts), titanium dioxide; zinc oxide; polysiloxane copolymer with a random distribution according to the formula:

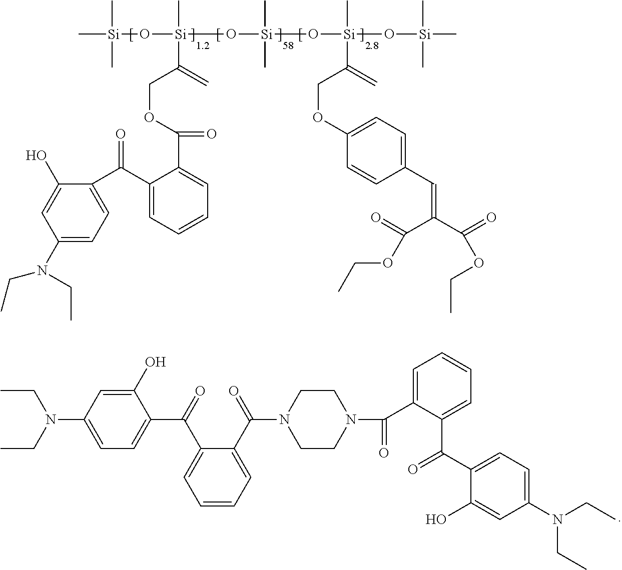

According to the invention, it is advantageous if the preparation according to the invention is free from p-methylbenzylidenecamphor.

According to the invention, it is advantageous if the preparation comprises starch and/or derivatives thereof. Here, the use of tapioca is preferred according to the invention.

According to the invention, starch and/or derivatives thereof are used here advantageously in an amount of from 0.05 to 8% by weight and preferably in an amount of from 0.1 to 5% by weight, based on the total weight of the preparation.

According to the invention, it is advantageous if the preparation comprises one or more compounds selected from the group of the compounds alpha-lipoic acid, folic acid, phytoene, D-biotin, coenzyme Q10, alpha-glucosylrutin, carnitine, carnosine, natural and/or synthetic isoflavonoids, flavonoids, creatine, creatinine, taurine, β-alanine, tocopheryl acetate, dihydroxyacetone; 8-hexadecene-1,16-dicarboxylic acid, glycerylglycose, (2-hydroxyethyl)urea, vitamin E and its derivatives and/or licochalcone A.

Active ingredients of this type can be used advantageously in an individual concentration of from 0.01 to 10% by weight, based on the total weight of the preparation.

In addition, the preparations according to the invention can advantageously also comprise self-tanning substances, such as, for example, dihydroxyacetone and/or melanin derivatives in concentrations of from 1% by weight up to 10% by weight, based on the total weight of the preparation.

In addition, the preparations according to the invention can advantageously also comprise repellents for protecting against flies, ticks and arachnids and the like. Advantageous examples are N,N-diethyl-3-methylbenzamide (trade name: meta-delphene, “DEET”), dimethyl phthalate (trade name: palatinol M, DMP), 2-(2-hydroxyethyl)-1-methylpropyl 1-piperidinecarboxylate and also in particular ethyl 3-(N-n-butyl-N-acetylamino)propionate (available under the trade name Insect Repellent® 3535 from Merck). The repellants can either be used individually or in combination.

Particularly advantageous preparations are also obtained if antioxidants are used as additives or active ingredients. According to the invention, the preparations advantageously comprise one or more antioxidants. Favorable, but nevertheless optional, antioxidants which can be used are all antioxidants customary or suitable for cosmetic applications.

Such antioxidants are advantageously selected from the group consisting of amino acids (e.g. glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and also salts thereof, dilauryl thiodipropionate, distearyl thiodipropionate, thiodipropionic acid and derivatives thereof (esters, ethers, peptides, lipids, nucleotides, nucleosides and salts) and also sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), also (metal) chelating agents (e.g. α-hydroxy fatty acids, palmitic acid, phytic acid, phytin, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, tocopherols and derivatives (e.g. vitamin E acetate), vitamin A and derivatives (vitamin A palmitate), and coniferyl benzoate of benzoin resin, rutinic acid and derivatives thereof, butylhydroxytoluene, butylhydroxyanisole, nordihydroguaiacic acid, nordihydroguaiaretic acid, trihydroxybutyrophenone, uric acid and derivatives thereof, mannose and derivatives thereof, sesamol, sesamolin, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) suitable according to the invention of these specified active ingredients.

The amount of the aforementioned antioxidants (one or more compounds) in the preparations is preferably 0.001 to 30% by weight, particularly preferably 0.05-20% by weight, in particular 1-10% by weight, based on the total weight of the preparation.

If vitamin E and/or derivatives thereof are the additional antioxidant or antioxidants, it is advantageous to select their particular concentrations from the range 0.001-10% by weight, based on the total weight of the formulation.

If vitamin A, or vitamin A derivatives, or carotenes or derivatives thereof are the additional antioxidant or antioxidants, it is advantageous to select their particular concentrations from the range 0.001-10% by weight, based on the total weight of the formulation.

According to the invention, it is advantageous if the preparation comprises one or more perfume substances selected from the group of the compounds limonene [5989-27-5], citral, linalool [78-70-6], alpha-isomethylionone [1335-46-2]. geraniol [106-24-1], citronellol [106-22-9], [24851-98-7], [18479-58-8], [54464-57-2], [80-54-6], [1222-05-5], [32388-55-9], [105-95-3], [31906-04-4], [8008-57-9], [32210-23-4], [120-57-0], [115-95-7], [101-86-0], [140-11-4], [6259-76-3] and [127-51-5], 2-isobutyl-4-hydroxy-4-methyltetrahydropyran, 2-tert-pentylcyclohexyl acetate, 3-methyl-5-phenyl-1-pentanol, 7-acetyl-1,1,3,4,4,6-hexamethyltetralin, adipic acid diester, alpha-amylcinnamaldehyde, alpha-methylionone, amyl C, butylphenylmethylpropional cinnamal, amyl salicylate, amyl cinnamyl alcohol, anise alcohol, benzoin, benzyl alcohol, benzyl benzoate, benzyl cinnamate, benzyl salicylate, bergamot oil, bitter orange oil, butylphenylmethylpropioal, cardamom oil, cedrol, cinnamal, cinnamyl alcohol, citronellylmethyl crotonate, citrus oil, coumarin, diethyl succinate, d-limonene, ethyllinalool, eugenol, evernia furfuracea extract, evernia prunastri extract, farnesol, guaiac wood oil, hexylcinnamal, hexyl salicylate, hydroxycitronellal, hydroxyisohexyl 3-cyclohexanecarboxaldehyde, lavender oil, lemon oil, linayl acetate, mandarin oil, menthyl PCA, methylheptanone, nutmeg oil, rosemary oil, sweet orange oil, terpineol, tonka bean oil, triethyl citrate and/or vanillin.

According to the invention, it advantageous here if the preparation according to the invention comprises one or more perfume substances in a total concentration of from 0.001 to 1% by weight, based on the total weight of the preparation.

According to the invention, it is preferred here if the preparation according to the invention comprises one or more perfume substances in a total concentration of from 0.001 to 0.5% by weight, based on the total weight of the preparation.

According to the invention, the preparation according to the invention advantageously comprises one or more compounds selected from the group of the compounds isopropanol, propylene glycol, ethylene glycol, ethylene glycol monoethyl or monobutyl ether, propylene glycol monomethyl, monoethyl or monobutyl ether, diethylene glycol monomethyl or monoethyl ether and analogous products, also alcohols of low carbon number, e.g. ethanol, isopropanol, 1,2-propanediol, 2-methyl-1,3-propanediol; 1,2-pentanediol; 1,2-hexanediol, 1,2-octanediol.

According to the invention, it is preferred if the preparation comprises one or more diols selected from the group of the compounds 2-methyl-1,3-propanediol, pentane-1,2-diol, hexane-1,2-diol, heptane-1,2-diol, octane-1,2-diol, nonane-1,2-diol, decane-1,2-diol.

According to the invention, diols of this type may advantageously be present in a concentration of from 0.1 to 20% by weight, based on the total weight of the preparation.

According to the invention, it is advantageous if the preparation comprises one or more parabens (e.g. methylparaben, ethylparaben, propylparaben, butylparaben).

Here, a total paraben content of from 0.05 to 1% by weight, based on the total weight of the preparation, is advantageous according to the invention.

According to the invention, the preparation according to the invention is preferably free from polyethylene glycols, i.e. is PEG-free.

Humectants (moisturizers) is the term used to refer to substances or substance mixtures which give cosmetic preparations the property, following application and/or spreading on the skin surface, of reducing the moisture loss by the horny layer (also called transepidermal water loss (TEWL)) and/or of positively influencing hydration of the horny layer.

Advantageous humectants (moisturizers) within the context of the present invention are, for example, glycerol, lactic acid and/or lactates, in particular sodium lactate, butylene glycol, propylene glycol, biosaccharide gum-1, glycine soya, ethylhexyloxyglycerol, pyrrolidonecarboxylic acid and urea. Furthermore, it is particularly advantageous to use polymeric moisturizers from the group of the water-soluble and/or water-swellable and/or water-gellable polysaccharides. Of particular advantage are, for example, hyaluronic acid, chitosan and/or a fucose-rich polysaccharide, which is listed in Chemical Abstracts under the registration number 178463-23-5 and is available e.g. under the name Fucogel® 1000 from SOLABIA S.A. Moisturizers can advantageously also be used as antiwrinkle active ingredients for protecting against skin changes, as arise e.g. during skin aging.

It is advantageous within the context of the present invention if the preparation according to the invention comprises one or more humectants in a total concentration of from 0.1 to 20% by weight and preferably in a total concentration of from 0.5 to 10% by weight, in each case based on the total weight of the preparation.

The cosmetic preparations according to the invention can also advantageously, although not necessarily, comprise fillers, which e.g. further improve the sensory and cosmetic properties of the formulations and, for example, bring about or enhance a velvety or silky skin feel. Advantageous fillers within the context of the present invention are starch and starch derivatives (such as e.g. tapioca starch, distarch phosphate, aluminum or sodium starch octenylsuccinate and the like), pigments which have neither primarily a UV filter effect nor a coloring effect (such as e.g. boron nitride etc.) and/or Aerosils® (CAS No. 7631-86-9) and/or talc.

The water phase of the preparations according to the invention can advantageously comprise customary cosmetic auxiliaries, such as, for example, alcohols, in particular those of low carbon number, preferably ethanol and/or isopropanol or polyols of low carbon number, and also ethers thereof, preferably propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, polymers, foam stabilizers, electrolytes, self-tanning agents, and also in particular one or more thickeners, which may be advantageously selected from the group consisting of silicon dioxide, aluminum silicates, polysaccharides or derivatives thereof, e.g. hyaluronic acid, xanthan gum, hydroxypropylmethylcellulose, particularly advantageously from the group of polyacrylates, preferably a polyacrylate from the group of so-called Carbopols, for example Carbopol grades 980, 981, 1382, 2984, 5984, in each case individually or in combination. Further thickeners advantageous according to the invention are those with the INCI name Acrylates/C10-30 Alkyl Acrylate Crosspolymer (e.g. Pemulen TR 1, Pemulen TR 2, Carbopol 1328 from NOVEON), and also Aristoflex AVC (INCI: Ammonium Acryloyldimethyltaurate/VP Copolymer).

According to the invention, the preparation according to the invention advantageously comprises film formers. Within the context of the present invention, film formers are substances of differing composition which are characterized by the following property: if a film former is dissolved in water or other suitable solvents and the solution is then applied to the skin, then it forms, following evaporation of the solvent, a film which essentially serves to fix the photofilters to the skin and in so doing to increase the water resistance of the product.

It is particularly advantageous to select film formers from the group of polymers based on polyvinylpyrrolidone (PVP)

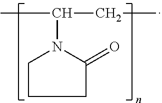

Particular preference is given to copolymers of polyvinylpyrrolidone, for example the PVP hexadecene copolymer and the PVP eicosene copolymer, which are available under the trade names Antaron V216 and Antaron V220 from GAF Chemicals Cooperation.

Further polymeric film formers, such as, for example, sodium polystyrenesulfonate, which is available under the trade name Flexan 130 from National Starch and Chemical Corp., and/or polyisobutene, obtainable from Rewo under the trade name Rewopal PIB 1000, are likewise advantageous. Further suitable polymers are e.g. polyacrylamides (Seppigel 305), polyvinyl alcohols, PVP, PVP/VA copolymers, polyglycols, acrylate/octylacrylamide copolymer (Dermacryl 79). The use of hydrogenated castor oil dimer dilinoleate (CAS 646054-62-8, INCI Hydrogenated Castor Oil Dimer Dilinoleate), which can be obtained from Kokyu Alcohol Kogyo under the name Risocast DA-H, or else PPG-3 benzyl ether myristate (CAS 403517-45-3), which can be obtained under the trade name Crodamol STS from Croda Chemicals, is likewise advantageous.

The oil phase of the preparation according to the invention is advantageously selected from the group of polar oils, for example from the group of lecithins and the fatty acid triglycerides, namely the triglycerol esters of saturated and/or unsaturated, branched and/or unbranched alkanecarboxylic acids of chain length from 8 to 24, in particular 12 to 18, carbon atoms.

The fatty acid triglycerides can, for example, be advantageously selected from the group of synthetic, semisynthetic and natural oils, such as e.g. cocoglyceride, olive oil, sunflower oil, jojoba oil, soybean oil, peanut oil, rapeseed oil, almond oil, palm oil, coconut oil, castor oil, wheatgerm oil, grapeseed oil, safflower oil, evening primrose oil, macadamia nut oil and the like.

Also advantageous according to the invention are e.g. natural waxes of animal and vegetable origin, such as, for example, beeswax and other insect waxes, and also berry wax, shea butter and/or lanolin (wool wax).

Within the context of the present invention, further advantageous polar oil components can also be selected from the group of the esters of saturated and/or unsaturated, branched and/or unbranched alkanecarboxylic acids of chain length from 3 to 30 carbon atoms and saturated and/or unsaturated, branched and/or unbranched alcohols of chain length from 3 to 30 carbon atoms, and also from the group of the esters of aromatic carboxylic acids and saturated and/or unsaturated, branched and/or unbranched alcohols of chain length from 3 to 30 carbon atoms. Such ester oils can then advantageously be selected from the group consisting of phenethyl benzoate, 2-phenylethyl benzoate, isopropyl lauroyl sarcosinate, phenyltrimethicone, cyclomethicone, dibutyl adipate, octyl palmitate, octyl cocoate, octyl isostearate, octyldodecyl myristate, octyldodecanol, cetearyl isononanoate, isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, stearyl heptanoate, oleyl oleate, oleyl erucate, erucyl oleate, erucyl erucate, tridecyl stearate, tridecyl trimellitate, and also synthetic, semisynthetic and natural mixtures of such esters, e.g. jojoba oil.

Furthermore, the oil phase can advantageously be selected from the group of dialkyl ethers and dialkyl carbonates, e.g. dicaprylyl ether (Cetiol OE) and/or dicaprylyl carbonate, for example that available under the trade name Cetiol CC from Cognis, are advantageous.

It is also preferred the oil component or components from the group consisting of isoeicosane, neopentyl glycol diheptanoate, propylene glycol dicaprylate/dicaprate, caprylic/capric diglyceryl succinate, butylene glycol dicaprylate/dicaprate, C₁₂₋₁₃-alkyl lactate, di-C₁₂₋₁₃-alkyl tartrate, triisostearin, dipentaerythrityl hexacaprylate/hexacaprate, propylene glycol monoisostearate, tricaprylin, dimethyl isosorbide. It is particularly advantageous if the oil phase of the formulations according to the invention has a content of C₁₂₋₁₅-alkyl benzoate or consists entirely of this.

Advantageous oil components are also e.g. butyloctyl salicylate (for example that available under the trade name Hallbrite BHB from CP Hall), tridecyl salicylate (which is available under the trade name Cosmacol ESI from Sasol), C12-C15 alkyl salicylate (available under the trade name Dermol NS from Alzo), hexadecyl benzoate and butyloctyl benzoate and mixtures thereof (Hallstar AB) and/or diethylhexyl naphthalate (Hallbrite TQ or Corapan TQ from Symrise).

Any desired mixtures of such oil and wax components can also be used advantageously within the context of the present invention.

Furthermore, the oil phase can likewise advantageously also comprise nonpolar oils, for example those which are selected from the group of branched and unbranched hydrocarbons and hydrocarbon waxes, in particular mineral oil, vaseline (petrolatum), paraffin oil, squalane and squalene, polyolefins, hydrogenated polyisobutenes, C13-16 isoparaffin and isohexadecane. Among the polyolefins, polydecenes are the preferred substances.

The preparations according to the invention can also advantageously comprise one or more substances from the following group of siloxane elastomers, for example in order to increase the water resistance and/or the sun protection factor of the products:
(a) siloxane elastomers which contain the units R₂SiO and RSiO_{1.5} and/or R₃SiO_{0.5} and/or SiO₂,
   where the individual radicals R are in each case independently of one another hydrogen, C₁₋₂₄-alkyl (such as, for example, methyl, ethyl, propyl) or aryl (such as, for example, phenyl or tolyl), alkenyl (such as, for example, vinyl) and the weight ratio of the units R₂SiO to RSiO_{1.5} is selected from the range from 1:1 to 30:1;
(b) siloxane elastomers which are insoluble and swellable in silicone oil and which are obtainable by the addition reaction of an organopolysiloxane (1) which contains silicon-bonded hydrogen with an organopolysiloxane (2) which contains unsaturated aliphatic groups,
   where the quantitative fractions used are chosen such that the amount of the hydrogen in the organopolysiloxane (1) or of the unsaturated aliphatic groups in the organopolysiloxane (2)
   is in the range from 1 to 20 mol % if the organopolysiloxane is not cyclic and
   is in the range from 1 to 50 mol % if the organopolysiloxane is cyclic.

Within the context of the present invention, the siloxane elastomer or elastomers are advantageously present in the form of spherical powders or in the form of gels.

Siloxane elastomers that are present in the form of spherical powders and advantageous according to the invention are those with the INCI name Dimethicone/Vinyl Dimethicone Crosspolymer, for example that available from DOW CORNING under the trade name DOW CORNING 9506 Powder.

It is particularly preferred if the siloxane elastomer is used in combination with oils composed of hydrocarbons of animal and/or vegetable origin, synthetic oils, synthetic esters, synthetic ethers or mixtures thereof.

According to the invention, the cosmetic preparations can comprise cosmetic auxiliaries, as are customarily used in such preparations, e.g. preservatives, preservation aids, complexing agents, bactericides, perfumes, substances for preventing or increasing foaming, dyes, pigments which have a coloring effect, thickeners, moisturizing and/or humectant substances, fillers which improve the skin feel, fats, oils waxes or other customary constituents of a cosmetic or dermatological formulation such as alcohols, polyols, polymers, foam stabilizers, electrolytes, organic solvents or silicone derivatives.

Within the context of the present invention, preparations for skincare are advantageous: they can serve for cosmetic sun protection, and also as a make-up product in decorative cosmetics.

Depending on their formulation, cosmetic compositions within the context of the present invention can be used, for example, as skin protection cream, day cream or night cream etc. In some instances, it is possible and advantageous to use the compositions according to the invention as a basis for pharmaceutical formulations.

In particular, the use of the preparation according to the invention for protection against skin aging (in particular for protection against UV-induced skin aging), and also as sunscreen composition, is in accordance with the invention.

According to the invention, the preparation according to the invention advantageously has a pH of from 5 to 8. This can be established by means of the conventional acids, bases and buffer systems.

The use of the preparation in cosmetic sprays (in particular sunscreen compositions) is in accordance with the invention.

The use for the impregnation of cosmetic wipes is in accordance with the invention.

### COMPARATIVE EXPERIMENTS

| | A | B | C | D |
|---|---|---|---|---|
| Raw material | m [%] | m [%] | m [%] | m [%] |
| Sodium stearoyl glutamate | 0.2 | 0.2 | 0.2 | 0.2 |
| Copolymer of vinylpyrrolidone and acrylic | 0.6 | 0.8 | 1.0 | 1.2 |
| acid | | | | |
| Alcohol | 2.0 | 2.0 | 2.0 | 2.0 |
| Bis-ethylhexyloxyphenol | 1.0 | 1.0 | 1.0 | 1.0 |
| methoxyphenyltriazine | | | | |
| Butylmethoxydibenzoylmethane | 4.5 | 4.5 | 4.5 | 4.5 |
| Polysilicone-15 | 2.0 | 2.0 | 2.0 | 2.0 |
| Cyclomethicone | 4.0 | 4.0 | 4.0 | 4.0 |
| C₁₂₋₁₅ Alkyl benzoate | 4.0 | 4.0 | 4.0 | 4.0 |
| Perfume | 0.4 | 0.4 | 0.4 | 0.4 |
| Glycerol | 4.0 | 4.0 | 4.0 | 4.0 |
| Octocrylene | 9.0 | 9.0 | 9.0 | 9.0 |
| Phenylbenzimidazole sulfonic acid | 3.0 | 3.0 | 3.0 | 3.0 |
| Sucrose polystearate + hydrogenated | 1.0 | 1.0 | 1.0 | 1.0 |
| polyisobutene | | | | |
| Trisodium EDTA | 1.0 | 1.0 | 1.0 | 1.0 |
| VP/hexadecene copolymer | 0.5 | 0.5 | 0.5 | 0.5 |
| Preservative | 0.5 | 0.5 | 0.5 | 0.5 |
| Water | ad 100.0 | ad 100.0 | ad 100.0 | ad 100.0 |
| Viscosity mPas at 25° C. (Haake VT02) | 500 | 1500 | 3000 | 4300 |
| Stability after storage for 3 months at 40° C. | very | very | very | very |
| | good | good | good | good |

Moreover, emulsion A can be sprayed upside-down with the help e.g. of Seaquist pump PZ2 190DL USD1.

### EXAMPLES

The examples below are intended to illustrate the present invention without limiting it. Unless stated otherwise, all quantitative data, fractions and percentages are based on the weight and the total amount or on the total weight of the preparation.

| | Emulsion | | | | | |
|---|---|---|---|---|---|---|
| | 1 | 2 | 3 | 4 | 5 | 6 |
| Sodium stearoyl glutamate | 0.2 | 0.1 | 0.2 | 0.4 | 0.1 | 0.5 |
| Sucrose polystearate + | 1 | | 1 | 0.6 | 0.5 | |
| hydrogenated | | | | | | |
| polyisobutene | | | | | | |
| Copolymer of | 0.2 | 0.3 | 0.4 | 0.7 | 1 | 1.5 |
| vinylpyrrolidone and | | | | | | |
| acrylic acid | | | | | | |
| Acrylate/C10-30 alkyl | 0.3 | 0.2 | | | 0.2 | |
| acrylate crosspolymer | | | | | | |
| C₁₂₋₁₅ Alkyl benzoate | 5 | | | | 8 | 3 |
| Butylene glycol | 2 | | 7 | | | 3 |
| dicaprylate/dicaprate | | | | | | |
| Triheptanoin | 3 | | | | | |
| Octyldodecanol | | | 3 | | | |
| Dicaprylyl carbonate | 3 | 3 | | | | |
| Caprylic/capric triglyceride | | | | | 2 | |
| Isodecyl neopentanoate | | 2 | | | | |
| Cyclomethicone | | | 5 | 3 | | 2 |
| Dimethicone | | | | 2 | | |
| Glycerol | 5 | 2 | 5 | 7 | 1 | 5 |
| Bis-ethylhexyloxyphenol | 2 | 0.5 | 2 | 1 | | |
| methoxyphenyltriazine | | | | | | |
| Ethylhexyl triazine | 2.5 | 1 | | | 2 | |
| Butylmethoxydibenzoyl- | 4 | 0.5 | 5 | 3 | 2 | |
| methane | | | | | | |
| Diethylaminohydroxy- | | 3 | | | | 5 |
| benzoylhexyl benzoate | | | | | | |
| Polysilicone-15 | | | 3 | 2 | | |
| Ethylhexyl methoxycinnamate | 8 | 5 | 5 | | | |
| Octyl salicylate | | | 5 | | | 5 |
| Homosalate | | | | | 3 | |
| Octocrylene | | 3 | | 10 | 5 | |
| Phenylbenzimidazole sulfonic | | | 2 | 4 | | 3 |
| acid | | | | | | |
| Titanium dioxide | | | | | 2 | |
| Folic acid | 0.05 | | 0.1 | | 0.2 | |
| Ethanol | 2 | | 4 | | 3 | 2.5 |
| Perfume, preservative | q.s. | q.s. | q.s. | q.s. | q.s. | q.s. |
| Dyes, neutralizing agent etc. | q.s. | q.s. | q.s. | q.s. | q.s. | q.s. |
| Water | ad | ad | ad | ad | ad | ad |
| | 100.0 | 100.0 | 100.0 | 100.0 | 100.0 | 100.0 |
| | Example | | | | | |
| | 7 | 8 | 9 | 10 | 11 | 12 |
| Sodium stearoyl glutamate | 0.5 | 0.8 | 0.2 | 0.4 | 0.3 | 0.3 |
| Sucrose polystearate + | | | 1 | 0.8 | 0.5 | |
| hydrogenated | | | | | | |
| polyisobutene | | | | | | |
| Copolymer of | 1.5 | 0.8 | 1 | 0.5 | 0.5 | 0.3 |
| vinylpyrrolidone and | | | | | | |
| acrylic acid | | | | | | |
| Stearyl alcohol | | | | | 0.5 | |
| Cetyl alcohol | | 2 | | | | 2 |
| Acrylates/C₁₀₋₃₀ alkyl acrylate | | | | 0.3 | 0.2 | 0.1 |
| crosspolymer | | | | | | |
| Myristyl myristate | | | 1 | | | |
| Tapioca starch | 2 | | 4 | | 1 | |
| C₁₂₋₁₅ Alkyl benzoate | | 3 | 5 | | | |
| Butyleneglycol | 2 | 4 | | | | 3 |
| dicaprylate/dicaprate | | | | | | |
| Dicaprylyl carbonate | 4 | | 2 | | 2 | |
| Octyldodecanol | | | | | | 5 |
| Cocoglyceride | | 3 | | | | |
| Cyclomethicone | 3 | | | | 2 | |
| 2-Propylheptyl octanoate | 4 | 8 | 10 | 5 | 4 | 6 |
| Glycerol | 5 | 6 | 4 | 10 | 2 | 4 |
| Ethanol | 2 | | 1 | | | 4 |
| Butylmethoxydibenzoyl- | | 2 | 2 | | 3 | 4 |
| methane | | | | | | |
| Ethylhexyl methoxycinnamate | 5 | | | | | |
| Octyl salicylate | | | | | 5 | |
| Octocrylene | 5 | | | | | |
| Disodium phenylene-1,4- | 2 | | | 1.5 | | 2 |
| bis(2-benzimidazyl)-3,3′,5,5′- | | | | | | |
| tetrasulfonic acid salt | | | | | | |
| Phenylbenzimidazole | | 2 | 2 | | | 1 |
| sulfonic acid | | | | | | |
| Titanium dioxide | 1 | | | | | 0.5 |
| Dimethiconediethyl- | | 1 | 4 | | 0.5 | 3 |
| benzalmalonate | | | | | | |
| INCI: Polysilicon-15) | | | | | | |
| Ethylhexyltriazine | | | | 3 | | |
| Vitamin E acetate | 0.5 | 0.2 | 0.1 | 0.5 | 0.25 | 0.3 |
| Na₂H₂EDTA | 0.1 | 0.2 | 0.2 | 0.2 | 0.2 | 0.5 |
| Perfume | | | 0.2 | 0.1 | 0.3 | 0.25 |
| Preservative | q.s. | q.s. | q.s. | q.s. | q.s. | q.s. |
| Sodium hydroxide, dyes | q.s. | q.s. | q.s. | q.s. | q.s. | q.s. |
| Water | ad | ad | ad | ad | ad | ad |
| | 100.0 | 100.0 | 100.0 | 100.0 | 100.0 | 100.0 |

| | Emulsion | | | |
|---|---|---|---|---|
| | 13 | 14 | 15 | 16 |
| Sodium stearoyl glutamate | 0.5 | 0.2 | 0.25 | 0.4 |
| Glyceryl stearate citrate | | 1 | 0.5 | |
| Cetearyl alcohol | 2 | | | |
| Stearyl alcohol | | | 2 | |
| Cetyl alcohol | | | | 2.5 |
| Xanthan gum | | | 0.2 | |
| Copolymer of vinylpyrrolidone and acrylic | 0.5 | 0.2 | 0.1 | 0.7 |
| acid | | | | |
| Acrylates/C10-30 alkyl acrylate | | 0.2 | | |
| crosspolymer | | | | |
| Carbomer | | | | 0.3 |
| 2-Propylheptyl octanoate | 5 | 3 | 7 | 10 |
| C₁₂₋₁₅ alkyl benzoate | 2 | | | |
| Cocoglycerides | | 3 | | |
| Isopropyl palmitate | | | 4 | |
| Jojoba oil | | 2 | | 5 |
| Caprylic/capric triglyceride | 2 | | | |
| Almond oil | | | 3 | |
| 2-Methyl-1,3-propanediol | 4 | 2 | | |
| Glycerol | 2 | 5 | 10 | 5 |
| Shea butter | | 1 | | 2 |
| Sodium starch octenylsuccinate | 3 | | | |
| Tapioca starch | 1 | 2 | | |
| Alcohol | 1 | | 2 | |
| Creatine | 0.5 | | | |
| Coenzyme Q10 | | 0.1 | 0.2 | |
| Sodium ascorbyl phosphate | 0.5 | | | |
| Na₂H₂EDTA | 0.1 | 0.1 | 0.2 | |
| Methylparaben | 0.1 | 0.2 | 0.2 | 0.15 |
| Phenoxyethanol | 0.5 | 0.4 | 0.6 | 0.3 |
| Perfume | q.s. | q.s. | q.s. | q.s. |
| Dyes, etc. | q.s. | q.s. | q.s. | q.s. |
| Sodium hydroxide | q.s. | q.s. | q.s. | q.s. |
| Water | ad | ad | ad | ad |
| | 100.0 | 100.0 | 100.0 | 100.0 |

1-12. (canceled)

13. A cosmetic preparation, wherein the preparation comprises (a) sodium stearyl glutamate, and (b) a crosslinked copolymer of vinylpyrrolidone and acrylic acid.

14. The preparation of claim 13, wherein (b) comprises a compound with CAS No. 527685-31-0.

15. The preparation of claim 13, wherein (b) is present in a concentration of from 0.05% to 4% by weight, based on a total weight of the preparation.

16. The preparation of claim 13, wherein (a) is present in a concentration of from 0.05% to 2% by weight, based on a total weight of the preparation.

17. The preparation of claim 13, wherein the preparation further comprises sucrose polystearate.

18. The preparation of claim 17, wherein the sucrose polystearate is present in a concentration of from 0.1% to 6% by weight, based on a total weight of the preparation.

19. The preparation of claim 13, wherein the preparation further comprises hydrogenated polyisobutene.

20. The preparation of claim 19, wherein the hydrogenated polyisobutene is present in a concentration of from 0.05% to 2% by weight, based on a total weight of the preparation.

21. The preparation of claim 13, wherein the preparation further comprises one or more electrolytes.

22. The preparation of claim 13, wherein the preparation further comprises one or more UV filters.

23. The preparation of claim 13, wherein the preparation further comprises at least one of starch and a derivative thereof.

24. The preparation of claim 13, wherein the preparation further comprises one or more perfume substances.

25. The preparation of claim 13, wherein preparation further comprises one or more diols selected from 2-methyl-1,3-propanediol, pentane-1,2-diol, hexane-1,2-diol, heptane-1,2-diol, octane-1,2-diol, nonane-1,2-diol, decane-1,2-diol, and ethylhexylglycerol.

26. The preparation of claim 13, wherein preparation further comprises one or more parabens.

27. The preparation of claim 13, wherein the preparation is present as an emulsion.

28. The preparation of claim 27, wherein the preparation is present as an O/W emulsion.

29. The preparation of claim 13, wherein the preparation is sprayable.

30. A cosmetic O/W emulsion, wherein the emulsion comprises, based on a total weight of the emulsion,
(a) from 0.1% to 1% by weight of sodium stearyl glutamate, and
(b) from 0.1% to 3% by weight of a crosslinked copolymer of vinylpyrrolidone and acrylic acid.

31. The emulsion of claim 30, wherein the emulsion further comprises from 0.2% to 4% by weight of sucrose polystearate, based on a total weight of the emulsion.

32. The emulsion of claim 30, wherein the emulsion further comprises from 0.1% to 1% by weight of hydrogenated polyisobutene, based on a total weight of the emulsion.
